SEQUENCE LISTING (4 sequences)

SEQ ID NO: 1 - DNA, 20 bp, Plasmid pTL122
atcgacgagcatcctccatc

SEQ ID NO: 2 - DNA, 20 bp, Plasmid pTL122
tcaggcaaagcatgaagcag

SEQ ID NO: 3 - DNA, 24 bp, Plasmid pTL122
ttcgcccgccatgtcgcctacctg

SEQ ID NO: 4 - DNA, 24 bp, Plasmid pTL122
ggcgagggcgaacatcagcagggc

(19) United States
(12) Patent Application Publication
(10) Pub. No.: US 2009/0042236 A1
(43) Pub. Date: Feb. 12, 2009

(54) Method of identifying hydrogen evolving diazotrophic bacteria

(76) Inventors: Paul E. Bishop, Raleigh, NC (US); Telisa M. Loveless, Durham, NC (US); Jonathan Olson, Cary, NC (US); Jose M. Bruno-Barcena, Raleigh, NC (US)

Correspondence Address: USDA-ARS-OFFICE OF TECHNOLOGY TRANSFER;NATIONAL CTR FOR AGRICULTURAL UTILIZATION RESEARCH, 1815 N. UNIVERSITY STREET, PEORIA, IL, 61604, US

(21) Appl. No.: 12/218,929

(22) Filed: Jul. 18, 2008

Related U.S. Application Data
(60) Provisional application No. 60/959,940, filed on Jul. 18, 2007.

(51) Int. Cl. C12Q 1/02; C12N 1/20; C12P 3/00

(52) U.S. Cl. 435/29; 435/252.1; 435/168

(57) ABSTRACT

MohC is required for expression of uptake hydrogenase in A. vinelandii and inactivation of this gene results in H₂ evolution via nitrogenase 3. Since MohC⁻ mutants are tungsten tolerant under nitrogen-fixing conditions, tungsten can be used to select spontaneous tungsten-tolerant mutants of nitrogen-fixing bacteria that are genetically uncharacterized. A major advantage of producing H₂ via nitrogenase 3 is that carbon substrates used to culture the nitrogen-fixing bacteria harboring this nitrogenase are derived from plant sources.

### CROSS-REFERENCE TO RELATED APPLICATION

This present application claims priority under 35 U.S.C. § 119(e) to U.S. Provisional Ser. No. 60/959,940, which was filed on Jul. 18, 2007, the disclosure of which is hereby incorporated by reference.

### FIELD OF THE INVENTION

The present invention is a method for identifying mutant strains of nitrogen-fixing bacteria having the characteristic of evolving hydrogen under nitrogen-fixing conditions. Specifically under nitrogen-fixing conditions, tungsten is a selecting agent to identify hydrogen-evolving bacteria having genes encoding for nitrogenase 3.

### BACKGROUND OF INVENTION

Diazotrophic bacteria under nitrogen fixation conditions produce hydrogen as a byproduct of converting dinitrogen into ammonia. Most of the hydrogen formed is re-oxidized via an uptake hydrogenase enzyme, thus making nitrogen fixation an energy efficient process. Biological nitrogen-fixation requires a nitrogenase enzyme system to catalyze ATP-dependent reduction of dinitrogen to ammonia. There are three different nitrogenase systems that facilitate nitrogen fixation: a molybdenum-containing nitrogenase (nitrogenase 1), a vanadium-containing nitrogenase (nitrogenase 2), and an iron-only nitrogenase (nitrogenase 3) (Bishop, P. E., et al., 1992. Alternative nitrogen fixation systems, p. 736-762. In G. Stacey, R. H. Burris, and H. J. Evans (ed.), Biological nitrogen fixation. Chapman and Hall, New York, N.Y.). Depending on the bacteria and nitrogenase system, it remains a challenge to properly maintaining media. For instance, diazotrophs that utilize nitrogenase 1 are expressed in nitrogen-free media containing molybdenum; nitrogenase 2 is expressed in nitrogen-free, molybdenum-deficient media containing vanadium; nitrogenase 3 is express in nitrogen-free, molybdenum-deficient, and vanadium-deficient media (Loveless, T., et al., 1999. App. Environ. Microbiol. 65:4223-4226). While nitrogenase 1 system has been studied extensively for A. vinelandii, approximately 25% of reducing electrons into the reduction of protons to molecular hydrogen for nitrogenase 1 whereas nitrogenase 3 channels about 50% of its reducing electrons into the production of hydrogen gas (Bishop, P. E., et al., 1986. Biochem. J., 238:437-442).

Given the greater capacity for hydrogen evolution via nitrogenase 3, there is a need to effectively capture evolved hydrogen during nitrogenase 3 fixation conditions. However, it remains a challenge in the art to determine whether an anaerobic, aerobic, or phototrophic bacterium utilizes nitrogenase 3 and to further determine whether that bacterium has the ability to evolve hydrogen. As such, there is a need to quickly and efficiently identify hydrogen-evolving bacteria that utilize nitrogenase 3 given the bacterium's higher capacity for hydrogen evolution. By identifying and capturing evolved hydrogen, the captured hydrogen can be utilized as a commodity substitute for conventional fossil fuels (natural gas, coal, and petroleum). Additionally, utilization of biological organisms for hydrogen production has an advantage over conventional hydrogen production means inasmuch as the process such as photovoltaic splitting of water is an energy intensive process with respect to biological hydrogen production means.

Currently there is a focus on biological hydrogen production via nitrogen-fixing cyanobacteria and phototrophic bacteria. For example, a phototrophic hup⁻ mutant Rhodobacter capsulatus has the ability to process hydrogen via nitrogenase 1 and nitrogenase 3 as disclosed in Krahn, E., et al., 1996. Appl. Microbiol. Biotechnology, 46:285-290.

Another example is phototrophic bacterium for hydrogen production as disclosed in U.S. Pat. No. 5,804,424 wherein photosynthetic proteobacteria are cultured in an oxide or oxyanion medium and reducing said oxide or oxyanion. More specifically, photosynthetic Rhodobacter or Rhodopseudomonas bacteria are cultured in the presence of oxyanions tellurium and selenium, or oxide of europium and rhodium. However, utilizing photosynthetic bacteria presents a disadvantage inasmuch as culturing photosynthetic organisms requires a large surface area in order to capture sufficient solar energy for large scale hydrogen production. As such, there is a need identify heterotrophic, nitrogen-fixing bacteria that are not limited by the constraints of photosynthetic bacteria.

The diazotrophic bacterium Azotobacter vinelandii has been studied given the bacterium's ability to be grown aerobically and the fact that it posses three nitrogenases. Generally, tungsten inhibits nitrogen fixation in A. vinelandii (Keeler, R. F., et al., 1957. Arch. Biochem. Biophys., 70:585-590). A. vinelandii are aerobic soil bacteria capable of synthesizing molybdenum containing nitrogenase (nitrogenase 1) as well as synthesizing vanadium and iron nitrogenases (nitrogenase 2 and 3 respectively) depending on the presence or absence of molybdenum and vanadium. Wild-type strains of A. vinelandii generally cannot grow in N-free medium containing Na₂WO₄ inasmuch as both molybdenum and tungsten share a common transport system and tungsten inhibits molybdenum uptake (Premakumar, R., et al., 1996. J. Bacteriol., 178: 691-696). However, strains such as mutant strain A. vinelandii CA6 is able to grow diazotrophically in 1 mM Na₂WO₄ while appearing to utilize both nitrogenase 1 and nitrogenase 3 for diazotrophic growth (Premakumar, R., et al., 1996. J. Bacteriol., 178: 691-696). Additionally other A. vinelandii mutants, such as the WD2 mutant strain (derived from A. vinelandii ATCC 12837), have been described as having the ability to fix nitrogen in the presence of tungsten (Riddle, G. D., et al., 1982. J. Bacteriol., 152:72-80). However, the ability to utilize tungsten is unexpected inasmuch as the nitrogenase system for nitrogen fixation does not require tungsten as a requirement. Furthermore, tungsten is inhibitory to such wild type A. vinelandii stains such as CA and ATCC 12837.

Furthermore, identifying wild-type bacteria that utilize nitrogenase 3-dependent nitrogen fixation remains a challenge inasmuch as there is no means to quickly identify such bacteria. Current methods rely on utilizing an enrichment media devoid of molybdenum. However ensuring a molybdenum-free medium is a time-consuming and costly process. Alternatively, identifying nitrogenase 3 dependent bacteria requires sequencing its genome which is a time consuming process and require resorting to genetic engineering techniques such as inactivating hydrogen uptake genes. As such, there is a need for a quick method to identify nitrogen-fixing bacteria that utilize a plurality of nitrogenases systems, particularly nitrogenase 3, and a means to identify such environmental isolates that evolve hydrogen without the complication of utilizing molybdenum-free media or sequencing the genome of the organism.

### BRIEF SUMMARY OF THE INVENTION

Disclosed herein is a novel tungsten-tolerant mutant bacterial strain that evolves hydrogen. The mutation responsible for hydrogen evolution is located in an operon encoding a low affinity molybdenum transport system which in turn affects expression of the uptake hydrogenase. Furthermore, the invention provides a method for identifying a hydrogen-evolving bacterial strain, the method comprising selecting a nitrogen-fixing bacterial strain, culturing said strain on tungsten containing selection medium; and identifying hydrogen production strain by diazotrophic growth on said selection medium. The invention further provides methods of selecting organisms for desirable hydrogen-evolving phenotypes wherein the organism is selected via its ability to have tungsten-tolerance.

In another embodiment, the invention provides a method of producing hydrogen from diazotrophs, the method comprising supplying a bacteria, said bacteria containing a mutation in the gene mohC, wherein the mutation causes the organism to contain an inoperative uptake hydrogenase, contacting said bacteria with a carbon source, allowing said bacteria to metabolize said carbon source; and isolating and recovering hydrogen from said bacteria. Such carbon sources to be utilized are glucose, mannitol, maltose, sucrose, acetate, ethanol, glycerol, molasses, and sucrose.

### BRIEF DESCRIPTION OF THE DRAWING

The present invention together with the above and other objects and advantages may best be understood from the following detailed description of the embodiment of the invention illustrated in the drawings, wherein:

FIG. 1 is a physical map of the moh operon in A. vinelandii. The arrows show the genes and direction of transcription. Gene designations are shown below the arrows. Gene products are shown above the arrows. Dashed lines indicate deleted regions. Inverted triangles indicate interposon insertions. Strains carrying the mutations are shown on the right.

FIG. 2 is a graph depicting hydrogen accumulation (expressed as percentage of gas) in the head-space as a function of cultures grown on various carbon sources.

FIG. 3 is a graph of batch fermentation profile of aerated (30% pO₂) A. vinelandii CA culture grown in Burk medium at 30° C. on a bench-top reactor as a function of time. The graph depicts the evolution of the fermentation variables during the unrestricted growth and after pulsing the culture with 5 g/l Sucrose during stationary phase of growth.

FIG. 4 is a graph of batch fermentation profile of aerated (30% pO₂) A. vinelandii CA6 culture grown in Burk medium at 30° C. on a bench-top reactor as a function of time. Figure shows the evolution of the fermentation variables during the unrestricted growth and after pulsing the culture with 5 g/l sucrose during stationary phase of growth.

### DETAILED DESCRIPTION OF THE INVENTION

### Definitions

As used in the specification and claims, the singular form “a”, “an” and “the” include plural references unless the context clearly dictates otherwise. For example, the term “a cell” includes a plurality of cells, including mixtures thereof.

The term “diazotroph” is defined as bacteria that takes N₂ and converts it into nitrogen compounds such as ammonia. Aerobic diazotrophs include bacteria such as, but not limited to Azotobacter vinelandii, Azotobacter chroococcum, Azotobacter paspali, Azotobacter salinestris, and Azomonas macrocytogenes. It is contemplated that these diazotrophs utilizing carbon sources, including, but not limited to glucose, mannitol, maltose, sucrose, ethanol, glycerol, and molasses, would be identified by the disclosed method of identifying hydrogen evolution. These diazotrophs can be isolated from the soil or water bodies, or other environmental sources.

Furthermore, various diazotrophic bacteria are phototrophs and have the ability to obtain energy requirements via light energy, and inorganic electron source, and carbon dioxide as a carbon source. These bacteria, such as cyanobacteria, include but are not limited to Rhodobacter capsulatus, Anabaena variabilis, Rhodospirillum rubrum, Heliobacterium gestii, and Rhodopseudomonas palustris.

The term “selective agent” as defined herein is any substance that brings about differences in fertility or mortality for an organism. As applied to A. vinelandii, tungsten salts are utilized as a selective agent. Examples of such selective agent salts include, but are not limited sodium tungstate (Na₂WO₄), sodium tungstate dihydrate (Na₂WO₄*2H₂O), tungstic acid (H₂WO₄), silico tungstic acid gr (H₄SiW₁₂O₄₀*XH₂O), and dodeca-tungsto phosphoric acid (H₃PO₄*12WO₃*XH₂O).

The term “Klett” as defined herein is a unit of measurement of cell density based on the scattering of light by cells suspended in culture medium and it correlates with optical density based on light scattering. This unit of measurement is specific for measurements made using a Klett calorimeter. More specifically, a Klett unit as equal to 4×10⁶ colony-forming units per ml.

### Bacterial Strains and Media

A plurality of Azotobacter vinelandii strains (see Table 1) were grown in modified Burk medium without added Na₂MoO₄. When required, 1 μM Na₂MoO₄, 1 mM V₂O₅, or 1 mM Na₂WO₄ was added to the media. Na₂MoO₄ was purchased from Fisher Scientific, Georgia, USA while V₂O₅ and Na₂WO₄ were purchased from Alfa Aesar, Massachusetts, USA. Fixed nitrogen was added as ammonium acetate to a final concentration of 28 mM. When required, kanamycin was added at 10 μg/ml. Escherichia coli strain TOP10 was cultured in Luria-Bertani medium. When required, antibiotics were added to the following concentrations: kanamycin, 50 μg/ml; ampicillin, 50 μg/ml.

Several wild-types and strains of bacteria were grown aerobically in the presence of tungsten. The bacterial strains that were tested for tungsten resistance are listed in Table 1.

TABLE 1
Bacterial strains

| Bacterial Strains | Relevant genotype or properties | Reference |
|---|---|---|
| Escherichia coli | | |
| TOP10 | F⁻ mcrA Δ(mrr-hsdRMS-mcrBC) | Invitrogen |
| | Φ80lacZΔM15 ΔlacX74 recA1 | |
| | araD139 Δ(ara-leu)7697 galU galK | |
| | rpsL (Str^{R}) endA1 nupG | |
| K12 71-18 | supE thi Δ(lac-proAB) F′ (proAB⁺) | Maniatis, T. et al., 1982. |
| | lacI^{q}ZΔM15 | Molecular cloning: a |
| | | laboratory manual. Cold |
| | | Spring Harbor |
| | | Laboratory, Cold Spring |
| | | Harbor, NY |
| Azotobacter | | |
| vinelandii | | |
| CA | Wild type | Bush, J. A., et al., 1959. |
| | | Nature (London) 184: |
| | | 381-382. |
| CA6 | tungsten tolerant | Premakumar R., et al. |
| | | 1996. J. Bacteriol, |
| | | 178: 691-696. |
| CA125 | tungsten tolerant mohC::kan | Loveless, T., et al. 2008. |
| CA70-Wt | ΔanfHD | Joerger et al., J. |
| | | Bacteriol. 171: 1075-1086 |
| CA129 | tungsten tolerant mohB::kan | Loveless, T., et al. 2008. |
| CA130 | tungsten tolerant mohA::kan | Loveless, T., et al. 2008. |
| Environmental | | |
| isolates | | |
| Br5 | soil, Foz de Iguazu, Brazil | Betancourt, D., et al. |
| | | 2008. Appl. Environ. |
| | | Microbiol. 74: 3471-3480 |
| Br6 | soil, Foz de Iguazu, Brazil | Betancourt, D., et al. |
| | | 2008. Appl. Environ. |
| | | Microbiol. 74: 3471-3480 |
| Br7 | soil, Foz de Iguazu, Brazil | Betancourt, D., et al. |
| | | 2008. Appl. Environ. |
| | | Microbiol. 74: 3471-3480 |
| Mu7 | wood chip mulch, Durham, North | Betancourt, D., et al. |
| | Carolina | 2008. Appl. Environ. |
| | | Microbiol. 74: 3471-3480 |

Growth of A. vinelandii Strains

For growth experiments, Burk medium was inoculated to a cell density of approximately 15 Klett units where 1 Klett unit equals 4×10⁶ colony forming units per ml. Growth at 30° C. was monitored with a Klett-Summerson calorimeter equipped with a no. 66 filter (red).

Construction of A. vinelandii Strains Carrying Kan^{r} Cartridge Insertions

A. vinelandii cells were made competent and transformed as described by Page, W. J., and M. von Tigerstrom and incorporated herein by reference. (Page, W., and M. von Tigerstrom, 1979. J. Bacteriol., 139:1058-1061). Transformations were conducted by streaking the desired strain on a plate containing ammonium acetate but devoid of molybdenum. The plate was incubated for a period of two to three days at 28° C. Strains were then transferred to plates devoid of both molybdenum and iron. The plate was then incubated for a period of an additional two to three days at 28° C. wherein a thin bacterial film developed on the plate. A loopful of cells from the plate devoid of both molybdenum and iron is taken and resuspended in an eppendorf tube containing 300 μl of 1× Burks buffer, while the selected plasmids are added to the eppendorf tube and inverted. The tube would then settle for an hour at 28° C. and subsequently, cells from the eppendorf tube was transferred to a plate devoid of molybdenum and incubated overnight at 28° C. The following day, 1 ml of 1× Burks buffer was added to the plate and the cells were collected in a tube and prepared for selection on the selection media.

Competent cells were incubated for 20 minutes at 30° C. in buffer containing 8 mM magnesium sulfate. pPM116 carrying an insert with the wild-type allele(s) was partially digested with EcoRI (New England Biolabs) and ligated to a 1.3-kbp EcoRI fragment containing a kanamycin-resistance cartridge from pKISS (Pharmacia, Piscataway, N.J.). E. coli K12 71-18 cells were transformed with the ligation mixture and Kan^{r} Amp^{r} transformants were selected. The location of the kan interposon was confirmed by HindIII and SalI restriction endonuclease analysis, and one of the Kan^{r} recombinants, pPM117, was used to transform A. vinelandii strain CA. Kan^{r} Amp^{s} transformants (indicative of a double-crossover event) were transferred at least four times on medium containing kanamycin to ensure segregation of the kan interposon. The transformants were tested for the ability to grow diazotrophically in the presence of 1 mM Na₂WO₄ and one of the tungsten-tolerant transformants (designated CA125) was selected for further study.

Plasmid pTL120 containing mohA was created using forward primer 1107F 5′TTCGCCCGCCATGTCGCCTACCTG3′ and reverse primer 1107R 5′GGCGAGGGCGAACATCAGCAGGGC3′ yielding a 1992 bp PCR product. The product was cloned into Irivitrogen pCR®2.1® TOPO vector and DNA sequenced to verify insertion. Restriction enzyme BsmI was used to create a 125-bp deletion in pTL120 and Klenow treated to create blunt ends. The Kan^{r} cartridge was isolated from pKISS (Pharmacia) after PstI digestion and performing the Klenow procedure to fill the ends. This 1.3-kb Kan^{r} cartridge was ligated to the pTL120 resulting in pTL121. The Kan^{r} cartridge insertion was verified by individual restriction digests with XhoI and SmaI and further verified by DNA sequence. pTL121 was linearized with the restriction enzyme XcmI and used in the transformation of A. vinelandii strain CA. Kanamycin-resistant colonies were transferred at least four times on medium containing kanamycin to ensure segregation of the kan interposon before testing for the ability to grow diazotrophically on Burk medium containing 1 mM NaWO₄. One of the tungsten-tolerant transformants (designated CA130) was selected for further study. To verify the CA130 construct, genomic DNA was prepared, PCR amplified and DNA sequenced.

Plasmid pTL122 containing mohB was created using forward primer 1109F 5′ATCGACGAGCATCCTCCATC3′ and reverse primer 1109R 5′TCAGGCAAAGCATGAAGCAG3′ yielding a 1973 bp PCR product. This product was cloned into Invitrogen pCR®2.1® (TOPO vector and DNA sequenced to verify the insertion. pTL122 was digested with restriction enzymes HincII and EcoNI creating a 439-bp deletion, Klenow treated and ligated to the PstI digested pKISS kanamycin cartridge. The ends of the cartridge were made blunt by the Klenow procedure. The kan insertion was verified by individual restriction digests with AfeI, BstEI, SbfI, XhoI and SmaI and further verified by DNA sequence. The resulting pTL123 was linearized with the restriction enzyme XcmI and used in the transformation of A. vinelandii strain CA. Kanamycin-resistant colonies were transferred at least four times on medium containing kanamycin (10 μg/ml) to ensure segregation of the kan interposon before testing for the ability to grow diazotrophically on Burks medium containing 1 mM NaWO₄. One of the tungsten-tolerant transformants (designated CA129) was selected for further study. To verify the CA129 construct, genomic DNA was prepared, PCR amplified and DNA sequenced. The plasmids used are itemized in the following Table 2.

TABLE 2

| Plasmids | Relevant genotype or properties | Reference |
|---|---|---|
| pPM116 | Amp^{r}, 2.1-kbp SalI-SphI fragment | Loveless, T., et al. 2008. |
| | containing the wt allele from CA | |
| pKISS | Amp^{r} Kan^{r} pUC4 with Kan^{r} cartridge | Pharmacia |
| | derived from Tn903 | |
| pPM117 | Amp^{r}, same as pPM116 except the Kan^{r} | Loveless, T., et al. 2008. |
| | cartridge is inserted in a EcoRI site | |
| pCR ® 2.1-TOPO ® | 3.9kbp TOPO TA cloning vector | Invitrogen |
| pTL120 | Amp^{r} Kan^{r} 1.9-kbp pcr fragment | Photini |
| | containing the wt allele mohA | |
| pTL121 | Amp^{r} Kan^{r} same as pTL120 except with | Loveless, T., et al. 2008. |
| | kan^{r} cartridge insertion | |
| pTL122 | Amp^{r} Kan^{r} 1.9-kbp pcr fragment | Loveless, T., et al. 2008. |
| | containing the wt allele mohB | |
| pTL123 | Amp^{r} Kan^{r} same as pTL122 except with | Loveless, T., et al. 2008. |
| | kan^{r} cartridge insertion in | |

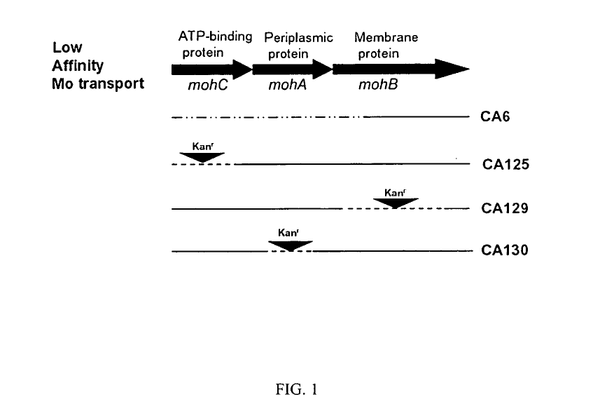

A. vinelandii strain CA6. During H₂ evolution tests the mutant strain A. vinelandii CA6, which carries mutations in genes that are thought to encode a low affinity molybdenum uptake system, evolved H₂. This was unexpected inasmuch as this strain is not known to have mutations in genes required for the uptake hydrogenase. Strain CA6 carries mutational alterations in all three of the genes that comprise an operon believed to encode a low affinity molybdenum uptake system. The genes, designated as mohCAB are thought to be part of a putative molybdenum uptake system, and as such, A. vinelandii strains were engineered containing interposon plus deletion mutations for each of the genes as shown in FIG. 1.

### DNA Methods

In vitro DNA manipulations, transformations, and restriction analysis were performed according to standard protocol known to those skilled in the art. Specifically, DNA fragments isolated from gels were done using QIAquick Gel Extraction Kit Protocol (Qiagen, Valencia, Calif.). Ligations were accomplished using the procedure of the Quick Ligation Kit (New England Biolabs Inc., Beverly, Mass.) or for TA cloning, Invitrogen TOPO TA Cloning® Kit (Invitrogen, Carlsbad, Calif.) was used. Tailing procedures were carried out according to Promega's (Madison, Wis.) tailing procedure in the pGEM®-T and pGEM®-T Easy Vector System technical manual. PCR amplifications were carried out using Invitrogen PCRx Enhancer System. PCR parameters were 30 cycles of 95° C. for 40 s, 55° C. for 30 s, and 1 min kb⁻¹ for 68° C.

### Selection for Tungsten-Tolerent A. Vinelandii Mutant Strains and Environmental Isolates

A. vinelandii strains and environmental isolates (Table 1) were grown on a Burk medium agar plate containing ammonium (for Nitrogen) and lacking molybdenum (Mo) were inoculated into liquid medium (30 ml) of the same composition to a cell density of 15 Klett units in a 300 ml side-arm flask. The carbon source used for A. vinelandii was 2% sucrose (wt/v). Each culture was grown overnight at 30° C. with vigorous shaking. Cells were centrifuged and washed with 30 ml of Nitrogen-free Burk liquid medium (lacking Mo). Each cell pellet was resuspended in 4 ml of Nitrogen-free Burk medium (lacking Mo). This suspension was used to inoculate 60 ml of N-free Burk medium (in a 500 ml side-arm flask), supplemented with either 1 μM Na₂MoO₄, 1 μM V₂O₅ or without added Na₂MoO₄, to a cell density of 15 Klett units. Quite surprisingly, it was discovered that A. vinelandii strains and environmental strains that were tungsten-tolerant mutants that are able to grow in the presence of Na₂WO₄ evolved hydrogen values close to that of strain CA6 and CA125. Growth of the culture was monitored until the cell density reached 50 to 70 Klett units.

After confirming that no measurable H₂ evolution was observed, each strain was spread on solid medium of the same liquid media referred to supra and supplemented with ammonium acetate (10 mM) and incubated overnight. The confluent culture was divided into sections. Each section was then used as inoculum for Burk glucose liquid medium with Na₂WO₄ added to a final concentration of 1 mM. Na₂WO₄ was selected inasmuch as the salt is water soluble. Other tungsten salts, such as sodium tungstate dihydrate (Na₂WO₄*2H₂O), tungstic acid (H₂WO₄), silico tungstic acid gr (H₄SiW₁₂O₄₀*XH₂O), dodeca-tungsto phosphoric acid (H₃PO₄*12WO₃*XH₂O) can also be used as selective agents. After the cultures reached a medium density (approximately 100 Klett units) usually after 3-5 days of incubation, a 10% inoculum of each culture was transferred into fresh medium followed by 4 to 5 subsequent transfers before testing for H₂ evolution. Dilutions were made of the cultures that were positive for H₂ evolution followed by plating for single colonies. After incubation for 30 minutes, 10 colonies were picked from each plate and tested for H₂ evolution.

To measure H₂, the 60 ml culture was divided equally between two 125 ml serum bottles. The bottles were sealed with a tight-fitting collared rubber stopper. The duplicate 30 ml cultures were placed in a New Brunswick shaker incubator set at 30° C. with shaking at 225 rpm. Gas headspace samples (3 ml) were withdrawn using a 3 ml plastic syringe at 0, 60 and 90 minute intervals from one of the duplicate cultures. Hydrogen was measured using a Shimadzu 17A gas chromatograph equipped with a TCD (thermal conductivity detector) and a 80/100 mesh Molecular Sieve 13× column (8 ft×¼ inch). Temperatures of the injector, detector, and column were 100, 120, and 50° C., respectively. The carrier gas was zero grade nitrogen set at a flow rate of 40 ml/min. To measure protein, 2 ml culture samples were withdrawn from the other duplicate 30 ml culture and transferred to a 2 ml flip-top microtube and placed on ice until samples were collected for all time intervals. After the culture samples for protein were collected, they were centrifuged, and the cell pellets were stored at −80° C. until the protein analyses were conducted.

Protein analysis was conducted according to the directions of the Pierce BCA Protein Assay Kit (Cat#23225). Briefly, working reagent was prepared by combining BCA reagent A with BCA reagent B at a 50:1 ratio. A 0.1 ml of the sample was combined with 2 ml of the working reagent and thoroughly mixed and incubated at 37° C. for 30 minutes and then cooled to room temperature. Absorbance of the sample was measured at 562 nm.

### Hydrogen Production by CA6 in a Bioreactor

Growth experiments were conducted in a 2 liter reactor model BIOSTAT B PLUS equipped with PID control units for pH, temperature, oxygen, and agitation speed (Sartorious, Germany). Experiments were performed with a working volume of 1000 ml maintained at 30° C. and the pH was allowed to vary during the experiments. Reactor and inoculum cultures were prepared with Burk medium containing 2% sucrose (w/v). Inoculum cultures were incubated at 30° C. with shaking (250 rpm). Reactor cultures were inoculated to a cell density of OD₆₀₀=0.1. The airflow through the reactor was set at 1 lpm of air at 1 atm. Dissolved oxygen, measured with an electrode (Mettler-Toledo, US) was maintained above 30% by slave control linked to the agitation speed between range limits of 300 to 1000 rpm using a PID control system. Cell growth was followed by on-line monitoring of the cell density. Exhaust reactor airflow was evaluated with a quadrupole mass spectrometer (QMS) Pfeiffer OmniStar®. The volumetric gas phase was analyzed for H₂, Ar, and CO₂ in real-time. In all of the determinations, the final concentrations were obtained by subtracting the amount of the compounds present in the air. Total compound mass was obtained by calculating the area under the production curves of H₂ and CO₂.

### EXAMPLE 1

### Tests for Hydrogen Evolution on Strains Containing Mutations in mohCAB

Table 3 shows the results of H₂ evolution measurements on strains containing mutations in the moh genes. Only strains CA6 and CA 125 result in H₂ evolution, leading to the conclusion that inactivation of mohC results in H₂ evolution while inactivation of either mohA or mohB does not result in H₂ evolution. mohC encodes the ATP-binding protein of the ABC cassette mohCAB while mohA and mohB encode the periplasmic and membrane spanning proteins, respectively (FIG. 1). Inactivation of any one of the three genes in the moh operon results in the ability of the mutant strain being able to grow in the presence of Na₂WO₄ under nitrogen-fixing conditions. This phenotype has previously been referred to as tungsten tolerant or tungsten resistant. Diazotrophic growth of strain CA6 in the presence of tungsten is due to expression of the nitrogenase 3. The results of hydrogen evolution for the listed strains are itemized in the following Table 3.

TABLE 3
Evolution of Hydrogen by A. vinelandii strains expressed
in μM H₂/minute/mg of protein

| Strain | —Mo | 1 μM V₂O₅ | 1 μM Na₂MoO₄ |
|---|---|---|---|
| CA | 0 | 0 | 0 |
| CA6 | 19.97^{a} | 20.16 | 10.61 |
| CA125 | 12.24 | 16.92 | 10.98 |
| CA129 | 0 | 0 | 0 |
| CA130 | 0 | 0 | 0 |

### EXAMPLE 2

### Growth and Hydrogen Production by A. Vinelandii Strains CA and CA6 in a Bench-Top Reactor

As a prelude to scaling up the H₂ production process, growth and H₂ production in a batch-fed culture were determined. Time-course growth experiments were performed in a bench-top reactor and the volumetric production of H₂ and CO₂ were quantified. The lot of medium and identical environmental conditions (temperature, pH, airflow, dissolved oxygen) for both CA (wild-type control) and CA6 remained consistent. Under these conditions, independent experiments were performed for each strain. The strains were grown with the carbon source as the limiting component and due to the high cellular oxygen demand the pO₂ set-point was set at 30%. To maintain the value of dissolved oxygen the airflow was maintained constant and variable agitation reached values of 1000 rpm exposing cells to increased shear.

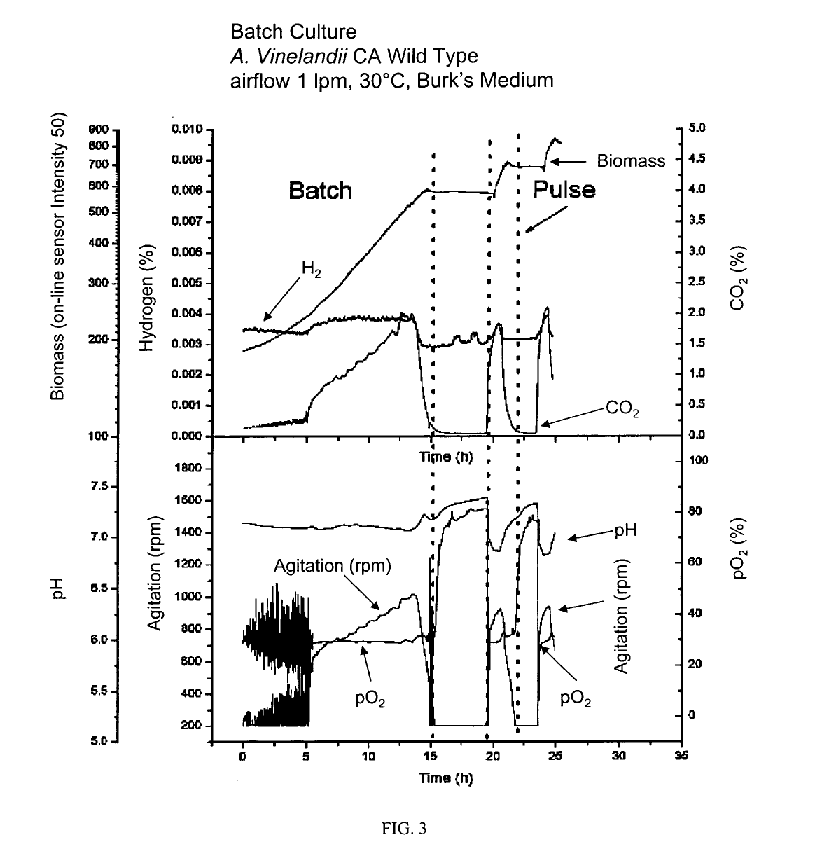

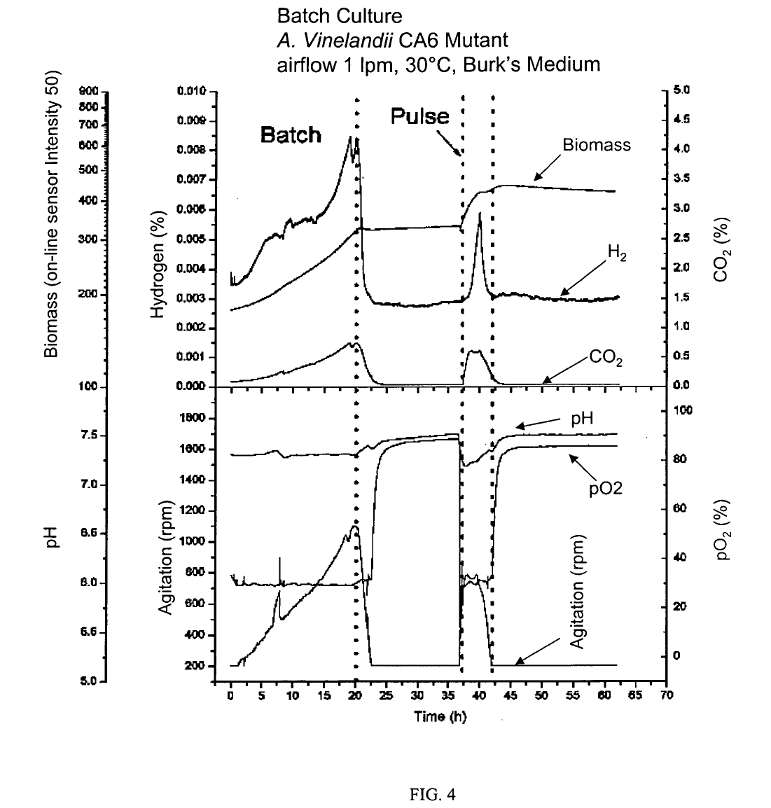

FIGS. 3 and 4 show culture variables monitored continuously during the growth experiments. Turbidity, cell dry weight, and parameters that define the strain growth were determined during the experiments and the relevant data are summarized in Table 4. The data show significant variations in the biomass yield and in the kinetic parameters of cell growth between the two strains. The energetic inefficiency of the H₂-producing strain (CA6) explains the divergence between the values obtained for the two strains. As expected, H₂ production is associated with growth and substrate assimilation. The substrate pulse (FIG. 3, 4) shows that H₂ production by the culture recovers even after 17.5 hours of substrate starvation. This observation is important because it indicates that cellular metabolism is robust and is able to recover after prolonged substrate starvation. The kinetic results for strains CA and CA6 are itemized in the following Table 4.

TABLE 4
Kinetic parameters and yields of Azotobacter vinelandii strains
CA and CA6 grown in Burk medium at 30° C. on
a bench-top reactor at 30% pO₂

| | Biomass Yield (Yx/s) | | |
|---|---|---|---|
| | mg/g | c-mol X**/mol | |
| Strain | C₁₂H₂₂O₁₁ | C₁₂H₂₂O₁₁ | μ (h⁻¹)* |
| CA | 41.46 | 0.55 | 0.10 |
| CA6 | 24.5 | 0.324 | 0.033 |

| | g CO₂/g | mol CO₂/mol | mol CO₂/ | mmol H₂/mol |
|---|---|---|---|---|
| Strain | C₁₂H₂₂O₁₁ | C₁₂H₂₂O₁₁ | mmol H₂ | C₁₂H₂₂O₁₁ |
| | Exponential Growth Phase | | | |
| | 20 g/L Sucrose limiting growth substrate | | | |
| | Hydrogen and CO₂ Yields | | | |
| CA | 0.687 | 5.340 | — | — |
| CA6 | 0.66 | 4.95 | 13.17 | 37.21 |
| | Stationary Phase Pulse | | | |
| | 5 g/L of Sucrose Limiting growth substrate | | | |
| | Hydrogen and CO₂ Yields | | | |
| CA | 0.48 | 3.80 | — | — |
| CA6 | 0.454 | 3.53 | 0.52 | 6.7 |

*Maximum specific growth rate (h⁻¹)
**X = Biomass;
***S = Sucrose

### EXAMPLE 3

### Isolation of Spontaneous Tungsten-Tolerant Mutants of Environmental Isolates Known to have Genes Encoding Nitrogenase 3

Since A. vinelandii strain CA6 is a spontaneous mutant strain that was originally isolated after prolonged incubation in the presence of 1 mM Na₂WO₄, it remained an open question as to whether tungsten-tolerant mutants of diazotrophic environmental isolates evolved hydrogen while using tungsten as a selection agent. As such, spontaneous tungsten-tolerant mutants from the environmental isolates Br5, Br6, Br7, and Mu7 were obtained from various sources (Table 1). The expectation was that tungsten-resistant strains would be a mixture of MohC⁻, MohA⁻ and MohB⁻ mutant cells. Therefore mutants were purified by single colony isolation and retested for H₂ evolution via the methods described supra. These mutants were designated as Br5-Wt, Br6-Wt, Br7-Wt, and Mu7-Wt (where Wt stands for tungsten tolerance). The results of hydrogen evolution for the environmentally isolated strains are itemized in the following Table 5.

TABLE 5
Hydrogen Evolution by Tungsten-tolerant Environmental Isolates

| | | Hydrogen | |
|---|---|---|---|
| | Total protein per | evolved | Rate of H₂ evolved |
| Strain | culture (mg) | per culture (ml) | (μM H₂/min/mg protein)^{a} |
| Br5-Wt | 3.710 ± 0.052^{b} | 20.004 ± 0.652 | 8.563 ± 0.138 |
| Br6-Wt | 2.384 ± 0.374 | 12.157 ± 1.167 | 9.003 ± 1.890 |
| Br7-Wt | 2.480 ± 0.531 | 15.642 ± 1.908 | 10.956 ± 3.485 |
| Mu7-Wt | 4.708 ± 0.396 | 21.582 ± 0.457 | 7.285 ± 0.470 |

^{a}Incubation time for all strains were 30 minutes.
^{b}Average ± standard deviation based on two independent experiments.

### EXAMPLE 4

### Effect of Carbon Substrates on Hydrogen Evolution by A. Vinelandii Strain CA6

Serum bottles (150 ml total volume) were prepared containing 30 ml carbon-free Burk medium. A carbon source was then added to each bottle. Mannitol, maltose, sucrose, glucose, citric acid, acetate and molasses were added to a final concentration of 2% (w/v). Ethanol, glycerol, and phenol were added to a final concentration of 0.5% (v/v). Each bottle was then inoculated with 0.1 ml of inoculum prepared from plate grown cultures that were harvested by swab into carbon and nitrogen free Burk medium, washed 1×, and resuspended to an optical density (600 nm) of ˜0.5 in the same media. The bottles were then capped with a rubber septum secured by an aluminum crimp seal. Cultures were incubated at 30° C. with shaking (200 rpm) for indicated times. An OmmiStar™ Gas Analyzer (Pfeiffer Vacuum, Asslar Germany) was utilized to measure the gas composition of the head space above the cultures. The gas analyzer uses a turbo-molecular pump coupled to a quadrapole mass spectrometer to identify gases based on molecular mass. Data were collected and percent H₂ was determined by comparison to a H₂ standard curve generated by using vacuum-sparged serum bottles to which various concentrations of H₂ had been added.

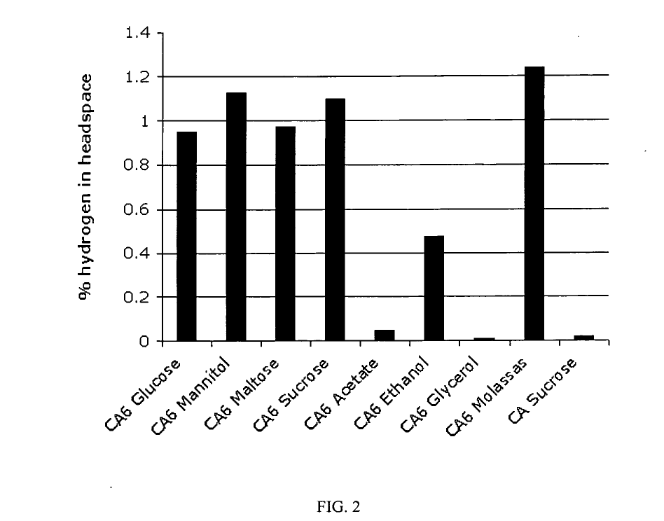

FIG. 2 shows the effect of a plurality of carbon substrates plus molasses on H₂ evolution by strain CA6. Significant growth (based on optical density) was observed for A. vinelandii CA6 when grown in Burk media using glucose, mannitol, maltose, sucrose, ethanol, glycerol, and molasses as carbon sources. On the other hand, no growth was observed under our experimental conditions when cultures were provided with citric acid, phenol, catechol, or acetate. In each case, H₂ evolution correlated with growth. These results show that strain CA6 is capable of utilizing carbon sources for H₂ production which include sugars and alcohols, making it suitable for growth on cheap carbon sources. For example, molasses is an inexpensive carbon source that is a by-product of processing sugarcane, sugar beets, citrus fruit, corn, and sorghum.

### EXAMPLE 5

### A. vinelandii Strain CA70 Cultured with Vanadium and Using Tungsten as a Selection Agent

In order to investigate whether tungsten could be utilized as a selection agent concurrently in culture with Vanadium, A. vinelandii strain CA70 ΔanfHD was transferred to N-free Burks media containing 1 μM V₂O₅ and 1 mM Na₂WO₄ 10 to 12 times or until growth reached a high density after inoculation. Strain CA70 is an engineered strain wherein the structural genes for mohCnitrogenase 3, anfHD, have been excised. From this culture, dilutions were made and spread on N-free agar plate medium containing 1 μM V₂O₅ and 1 mM Na₂WO₄. Twelve colonies were picked with sterile toothpicks and each toothpick containing the colony was inoculated in 20 ml of N-free Burk medium containing 1 μM V₂O₅ and 1 mM Na₂WO₄ in a 125 ml serum bottle. The 12 cultures were incubated two to three days and seven of the twelve cultures were positive for H₂ production. The results of hydrogen evolution for strain CA70 strain is itemized in the following Table 6.

TABLE 6
Hydrogen Evolution by Tungsten-tolerant Azotobacter vinelandii
mutant CA70 using V-nitrogenase

| | | Hydrogen | |
|---|---|---|---|
| | Total protein per | evolved | Rate of H₂ evolved |
| Strain | culture (mg) | per culture (ml) | (μM H₂/min/mg protein)^{a} |
| CA70-Wt | 2.719 ± 0.1956^{b} | 9.697 ± 2.389 | 5.080 ± 1.200 |

^{a}Incubation time was 90 minutes.
^{b}Average ± standard deviation based on two independent experiments.

While the invention has been described with reference to details of the illustrated embodiment, these details are not intended to limit the scope of the invention as defined in the appended claims. The embodiment of the invention in which exclusive property or privilege is claimed is defined as follows:

1. A method for selecting a hydrogen-evolving bacteria strain, the method comprising:
supplying a nitrogen-fixing bacteria strain,
culturing said strain on a growth medium,
applying a tungsten salt as a selection agent to the culture; and
identifying hydrogen production strain by diazotrophic growth on said culture.

2. The method as recited in claim 1 wherein the bacteria is from the genus Azotobacter vinelandii.

3. The method as recited in claim 1 wherein the tungsten salt is Na₂WO₄.

4. The method as recited in claim 1 wherein said tungsten salt is selected from the group consisting of sodium tungstate dihydrate (Na₂WO₄*2H₂O), tungstic acid (H₂WO₄), silico tungstic acid gr (H₄SiW₁₂O₄₀*XH₂O), and dodeca-tungsto phosphoric acid (H₃PO₄*12WO₃*XH₂O).

5. The method as recited in claim 1 wherein the bacteria metabolizes in an anaerobic atmosphere.

6. The method as recited in claim 1 wherein the bacteria metabolizes in an aerobic atmosphere.

7. The method as recited in claim 6 wherein the aerobic atmosphere is between approximately 20% to approximately 30% partial pressure of oxygen.

8. The method as recited in claim 6 wherein the bacteria metabolizes in a temperature selected from approximately 25 to 30 degrees Celsius.

9. The method as recited in claim 1 wherein the growth medium further comprises vanadium pentoxide.

10. A biologically pure culture of microorganism identified through the method as recited in claim 1.

11. A method of producing hydrogen from bacteria, the method comprising:
supplying a bacteria strain, said bacteria containing a mutation for the gene mohC,
wherein the mutation causes the organism to contain an inoperative uptake hydrogenase,
contacting said bacteria strain with a carbon source,
allowing said bacteria strain to metabolize said carbon source; and
isolating and recovering hydrogen from said bacteria strain.

12. The method as recited in claim 11 wherein the bacteria is of the genus Azotobacter vinelandii.

13. The method as recited in claim 11 wherein the carbon source comprises plant biomass.

14. The method as recited in claim 11 wherein the carbon source is selected from the group consisting of glucose, mannitol, maltose, sucrose, acetate, ethanol, glycerol, molasses, and sucrose.

15. The method as recited in claim 11 wherein the bacteria metabolizes in an aerobic atmosphere.

16. The method as recited in claim 15 wherein the aerobic atmosphere is between approximately 20% to approximately 30% partial pressure of oxygen.

17. The method as recited in claim 15 wherein the bacteria metabolizes in a temperature selected from approximately 25 to approximately 30 degrees Celsius.

18. The method as recited in claim 11 wherein the bacteria metabolizes in an anaerobic atmosphere.

19. The method as recited in claim 11 wherein the bacteria is selected from the group consisting of CA6, Br5-Wt, Br6-Wt, Br7-Wt, and Mu7-Wt.

20. A hydrogen-evolving tungsten-tolerant bacteria strain selected by a process comprising:
supplying a nitrogen-fixing bacteria strain,
culturing said nitrogen-fixing strain on a growth medium,
applying a tungsten salt as a selection agent to the culture; and
collecting surviving bacteria strain.

21. The process as recited in claim 20 wherein the nitrogen-fixing bacteria is from the genus Azotobacter vinelandii.

22. The process as recited in claim 20 wherein the tungsten salt is Na₂WO₄.

23. The process as recited in claim 20 wherein said tungsten salt is selected from the group consisting of sodium tungstate dihydrate (Na₂WO₄*2H₂O), tungstic acid (H₂WO₄), silico tungstic acid gr (H₄SiW₁₂O₄₀*XH₂O), and dodeca-tungsto phosphoric acid (H₃PO₄*12WO₃*XH₂O).

24. The process as recited in claim 20 wherein the bacteria metabolizes in an anaerobic atmosphere.

25. The process as recited in claim 20 wherein the bacteria metabolizes in an aerobic atmosphere.

26. The process as recited in claim 25 wherein the aerobic atmosphere is between approximately 20% to approximately 30% partial pressure of oxygen.

27. The process as recited in claim 25 wherein the bacteria metabolizes in a temperature selected from approximately 25 to 30 degrees Celsius.

28. The process as recited in claim 20 wherein the growth medium further comprises vanadium pentoxide.
